(19) United States
(12) Patent Application Publication
(10) Pub. No.: US 2024/0423982 A1
(43) Pub. Date: Dec. 26, 2024

(54) TREATMENT OR PREVENTION OF NEW-ONSET TYPE 1 DIABETES WITH BARICITINIB

(71) Applicant: Eli Lilly and Company, Indianapolis, IN (US)

(72) Inventors: Thomas W. KAY, Fitzroy (AU); Helen E. THOMAS, Fitzroy (AU)

(21) Appl. No.: 18/667,433

(22) Filed: May 17, 2024

Related U.S. Application Data
(60) Provisional application No. 63/503,588, filed on May 22, 2023.

(51) Int. Cl. A61K 31/517; A61K 9/00; A61K 38/28; A61K 45/06; A61P 3/10

(52) U.S. Cl. CPC A61K 31/517; A61K 9/0053; A61K 38/28; A61K 45/06; A61P 3/10

(57) ABSTRACT

The present invention provides methods of treating or preventing new-onset type 1 diabetes with baricitinib, either alone or in combination with insulin, insulin analogues and/or other immunomodulatory agent.

### FIELD OF THE INVENTION

The present invention relates to methods of treating or preventing new-onset type 1 diabetes with baricitinib, either alone or in combination with insulin, insulin analogues and/or other immunomodulatory agents.

### BACKGROUND OF THE INVENTION

Baricitinib is an approved medication that belongs to the pharmacological class of Janus kinase (JAK) inhibitors. Janus kinases are a family of four protein tyrosine kinases (JAK1, JAK2, JAK3, and tyrosine kinase 2 [TYK2]) that play a role in cytokine signal transduction. Baricitinib demonstrates selectivity for, and inhibition of, JAK1 and JAK2 with lower potency towards inhibition of JAK3 or TYK2. In isolated enzyme assays, baricitinib inhibited the activities of JAK1, JAK2, TYK2, and JAK3 with half-maximal inhibitory concentration values of 5.9, 5.7, 53, and >400 nM, respectively (Fridman J. S., et al., “Selective inhibition of JAK1 and JAK2 is efficacious in rodent models of arthritis: preclinical characterization of INCB028050”, J Immunol, 2010; 184 (9): 5298-5307). Janus kinases are enzymes that transduce intracellular signals from cell surface receptors for a number of cytokines and growth factors involved in hematopoiesis, inflammation, and immune function (e.g., interleukin [IL]-2, IL-6, IL-12, IL15, IL-23, interferons, and granulocyte-macrophage colony-stimulating factor) (O'Shea J. J, et al., “The JAK-STAT pathway: impact on human disease and therapeutic intervention” Annum Rev Med, 2015; 66:311-328). Within the intracellular signalling pathway, JAKs phosphorylate and activate signal transducers and activators of transcriptors (STATs), which activate gene expression within the cell. Baricitinib modulates these signalling pathways by partially inhibiting JAK1 and JAK2 enzymatic activity, reducing the phosphorylation and activation of STATs and reducing inflammation, cellular activation, and proliferation of key immune cells (O'Shea J. J., et al., “JAKs and STATs in immunity, immunodeficiency, and cancer”, N Engl J Med, 2013; 368 (2) 161-170). WO 2009/114512 discloses various azetidine and cyclobutane derivatives, including baricitinib, as JAK inhibitors.

Type 1 diabetes (T1D), also known as autoimmune diabetes, is a chronic disease characterized by insulin deficiency due to pancreatic β-cell loss. The main manifestation of T1DM is hyperglycemia, which is controlled through administration of insulin, and/or insulin analogues. Complications traditionally associated with T1D include macrovascular conditions, such as coronary heart disease, stroke and peripheral arterial disease, and microvascular conditions, including diabetic kidney disease, retinopathy and peripheral neuropathy. While the prevalence of these conditions is declining with improvements in the management of T1D, patients are becoming more susceptible to a different set of complications, such as cancer, infections, liver disease, functional/cognitive disabilities, and affective disorders (Tomic D, et al., “The burden and risks of emerging complications of diabetes mellitus” Nat Rev Endocrinol, 2022; 18:525-539). As such, death rates due to T1D remain high, and the cost of treating T1D, as well as treating its complications, continues to weigh heavily on both patients and healthcare systems.

A potential role of an experimental JAK1/JAK2 inhibitor in the reversal of autoimmune insulitis in non-obese diabetic (NOD) mouse model has been suggested (Trivedi P. M., et al., “Repurposed JAK1/JAK2 inhibitor reverses established autoimmune insulitis in NOD mice”, Diabetes, 2017, 66:1650-1660). However, as stated by the authors of this study, significant further work is required to test whether these results can be translated to the safe and efficacious treatment of T1D in humans.

There are currently no approved JAK inhibitors for the treatment and prevention of T1D. Teplizumab, an injectable humanized anti-CD3 monoclonal antibody, has recently gained FDA approval as the first agent for delaying the onset of stage 3 T1D in adults and pediatric patients eight years and older who currently have stage 2 T1D.

Despite the recent approval of teplizumab, there remains a need for disease-modifying monotherapies or adjunct therapies for the treatment or prevention of new-onset T1D. New therapies are required to preserve the insulin secretion capacity in pre-diabetic patients, delay the decline in insulin secretion capacity in patients with new-onset T1D, improve glucose control in patients with new-onset T1D and/or improve pancreatic β-cell function capacity in patients with new-onset T1D. These therapies should be efficacious, but also safe and easily administered.

### SUMMARY OF THE INVENTION

The present invention provides baricitinib, either alone or in combination with insulin, insulin analogues and/or other immunoregulatory agents, as a therapy for the treatment or prevention of new-onset T1D. In certain aspects of the invention, baricitinib is administered in preferred dosages, dosage regimens and formulations to provide maximum efficacy, safety, and ease of administration.

It is herein demonstrated that baricitinib has a positive effect on the preservation of insulin secretion capacity in new-onset type-1 diabetes patients over the course of 48 weeks. This effect was manifested by higher levels of measured C-peptide and lower requirement for insulin administration in the patients treated with baricitinib versus placebo. In addition, baricitinib was well tolerated by patients. These results show that oral administration of baricitinib may be employed as a safe, efficacious, and easy to administer disease-modifying agent for the treatment and prevention of new-onset type 1 diabetes.

### DETAILED DESCRIPTION OF THE INVENTION

The present invention provides a method of treating or preventing new-onset type 1 diabetes in a subject, comprising administering an effective amount of baricitinib, or a pharmaceutically acceptable salt thereof.

In an embodiment of the invention the new-onset type 1 diabetes is type 1 diabetes clinically diagnosed according to American Diabetes Association guidelines (ADA) within 1 year prior to treatment commencement.

In an embodiment of the invention the new-onset type 1 diabetes is type 1 diabetes clinically diagnosed according to American Diabetes Association guidelines (ADA) within 6 months prior to treatment commencement.

In an embodiment of the invention the new-onset type 1 diabetes is type 1 diabetes clinically diagnosed according to American Diabetes Association guidelines (ADA) within 100 days prior to treatment commencement.

In an aspect of the invention the method further comprises the administration of an effective amount of insulin, an insulin analogue and/or other immunomodulatory agents. Insulin analogues comprise rapid-acting and long-acting insulins, such as insulin lispro, insulin aspart, insulin glulisine, insulin glargine and insulin determir. Immunomodulatory agents include agents that induce tolerance, reduce antigen presentation of pancreatic beta cell antigens, inhibit activation of cytotoxic T cells, enhance T regulatory cell numbers or function, inhibit activation of cytotoxic natural killer T cells, reduce cytokine production or inflammation, are β-cell antagonists, or are checkpoint agonists (for other examples see Bluestone J. A., et al., “Immunotherapy: Building a bridge to a cure for type 1 diabetes”, Science, 2021, 373:510-516). Immunomodulatory agents can comprise JAK inhibitors other than baricitinib, TYK2 inhibitors such as deucravacitinib, anti-CD3 antibodies such as teplizumab and otelixizumab and anti-CD20 antibodies such as rituximab.

In an embodiment of the invention the amount of baricitinib, or a pharmaceutically acceptable salt thereof, is 0.5 to 10 mg, 0.5 to 8 mg, 0.5 to 6 mg, 0.5 to 4 mg, 1 to 4 mg, and 2 to 4 mg. In a preferred embodiment the amount of baricitinib, or a pharmaceutically acceptable salt thereof, is 1 to 4 mg. In another embodiment the amount of baricitinib, or a pharmaceutically acceptable salt thereof, is selected from 0.5 mg, 1 mg, 1.5 mg, 2 mg, 2.5 mg, 3 mg, 3.5 mg, 4 mg, and 4.5 mg, 5 mg, 5.5 mg, 6 mg, 6.5 mg, 7 mg, 7.5 mg, 8 mg, 8.5 mg, 9 mg, 9.5 mg, and 10 mg. In a preferred embodiment the amount of baricitinib, or a pharmaceutically acceptable salt thereof, is 1 mg. In a preferred embodiment the amount of baricitinib, or a pharmaceutically acceptable salt thereof, is 2 mg. In another preferred embodiment the amount of baricitinib, or a pharmaceutically acceptable salt thereof, is 4 mg. In a further embodiment, the amount of baricitinib, or a pharmaceutically acceptable salt thereof, is fixed throughout the treatment period. In another embodiment the amount of baricitinib or a pharmaceutically acceptable salt thereof, is varied throughout the treatment period.

In an embodiment of the invention baricitinib, or a pharmaceutically acceptable salt thereof, is administered orally. In a further embodiment baricitinib, or a pharmaceutically acceptable salt thereof, is administered once daily. In another embodiment baricitinib, or a pharmaceutically acceptable salt thereof, is administered twice daily. In an embodiment baricitinib, or a pharmaceutically acceptable salt thereof, is administered as a pharmaceutical composition comprising baricitinib or a pharmaceutically acceptable salt thereof with one or more pharmaceutically acceptable carriers, diluents, or excipients. In some embodiments the pharmaceutical composition is in a form selected from a pill, a tablet, a solution, and a suspension.

In an embodiment of the invention baricitinib is administered as the free base. In another embodiment baricitinib is administered as a pharmaceutically acceptable salt thereof. Pharmaceutically acceptable salts of baricitinib include baricitinib phosphate salt.

The present invention further provides baricitinib, or a pharmaceutically acceptable salt thereof for use in the treatment or prevention of new-onset type 1 diabetes.

The present invention also provides baricitinib, or a pharmaceutically acceptable salt thereof in sequential, separate, or simultaneous combination with one or more agent selected from insulin, an insulin analogue and/or other immunomodulatory agents for use in the treatment or prevention of new-onset type 1 diabetes.

### Definitions

As used herein, the terms “treating”, or “treatment” refer to inhibiting, delaying the progression of, stopping, or reversing the progression of a disease or condition. More specifically, “treating”, or “treatment” refer to delaying the decline in insulin secretion capacity in patients with new-onset T1D, improving glucose control in patients with new-onset T1D and improving pancreatic β-cell function capacity in patients with new-onset T1D. As used herein, the terms “preventing”, or “prevention” refer to preventing, or delaying the clinical diagnosis of a disease or condition in an individual who may be predisposed to or at risk of developing the disease or condition but does not yet experience or display the pathology or symptomatology of the disease or condition. As used herein, “type 1 diabetes clinically diagnosed according to American Diabetes Association (ADA) criteria” refers to type 1 diabetes clinically diagnosed using the criteria detailed in ElSayed N. A., et al., “2. Classification and Diagnosis of Diabetes: Standards of Care in Diabetes-2023”, Diabetes Care, 2023, 46 (Supplement 1), S19-S21. As used herein, the term “subject” refers to a human subject. As used herein, the term “effective amount” refers to the amount or dose of compound of the invention, or a pharmaceutically acceptable salt thereof which, upon single or multiple dose administration to the subject, provides the desired effect in the subject. As used herein, the term “baricitinib” refers to {1-(ethylsulfonyl)-3-[4-(7H-pyrrolo[2,3-d]pyrimidin-4-yl)-1H-pyrazol-1-yl]azetidin-3-yl}acetonitrile:

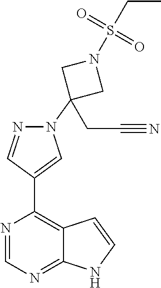 [SMILES: CCS(=O)(=O)N1CC(CC#N)(n2cc(-c3ncnc4[nH]ccc34)cn2)C1]

As used herein, the term “insulin analogue” is used to refer to synthetically produced variations of insulin that are different in amino acid sequence than that of native human insulin. As used herein, the term “insulin lispro” is used to refer to an insulin analogue which is identical to human insulin except for the transposition of proline and lysine at positions B28 and B29. As used herein, the term “insulin aspart” is used to refer to an insulin analogue which is identical to human insulin except for the replacement of proline with aspartic acid at position B28. As used herein, the term “insulin glulisine” is used to refer to an insulin analogue which is identical to human insulin except for the replacement of asparagine with lysine at position B3, and the replacement of lysine with glutamic acid at position B29. As used herein, the term “insulin glargine” is used to refer to an insulin analogue which is identical to human insulin except for the replacement of asparagine with glycine at position A21, and the extension of the B-chain by two arginine residues. As used herein, the term “insulin determir” is used to refer to an insulin analogue which is identical to human insulin except for the attachment of myristic acid to the lysine at position B29.

In the methods of the present invention, baricitinib or a pharmaceutically acceptable salt thereof is preferably formulated as a pharmaceutical composition administered by any route which makes it bioavailable. Most preferably, such compositions are for oral administration. Such pharmaceutical compositions and processes for preparing the same are well known in the art (See, e.g., Remington: The Science and Practice of Pharmacy, Adejare A., Editor, 23^{rd} Edition, Elsevier Academic Press, 2020).

In the methods of the present invention, baricitinib can be administered as a pharmaceutically acceptable salt thereof. Such pharmaceutically acceptable salt thereof can be formed, for example, by reaction of baricitinib free base with an appropriate pharmaceutically acceptable acid in a suitable solvent under standard conditions well known in the art (See, e.g., Berge S. M., et al., “Pharmaceutical Salts”, J. Pharm. Sci., 1977, 66:1-19).

### List of Abbreviations

| | |
|---|---|
| ADA | American Diabetes Association |
| AEs | adverse effects |
| CGM | continuous glucose monitoring |
| dL | deciliter |
| h | hour |
| HbA1c | glycated hemoglobin |
| kg | kilogram |
| L | liter |
| LDL | low-density lipoprotein |
| mg | milligram |
| min | minute |
| mmol | millimole |
| MMTT | mixed-meal tolerance test |
| p | probability |
| PBMC | peripheral blood mononuclear cells |
| pmol | picomole |
| SAEs | serious adverse effects |
| SEM | standard error of the mean |
| U | unit |

### BRIEF DESCRIPTION OF THE DRAWINGS

FIG. 1: Effect of baricitinib on (A) C-peptide, (B) insulin dose and (C) glycated haemoglobin level. (A) and (C) are shown as mean±SEM, (B) is shown as median [Q1, Q3]; * denotes p≤0.05, ** denotes p≤0.01, *** denotes p≤0.001.

FIG. 2: Glycaemic control as measured by continuous glucose monitor, represented by (A) coefficient of variation, (B) % time in range 70-180 mg/dl (3.9-10.0 mmol/L), (C) % time above range>180 mg/dL (>10.0 mmol/L), and (D) % time below range<70 mg/dL (<3.9 mmol/L). For each panel, mean±SEM is shown at each time point; * denotes p≤0.05, ** denotes p≤0.01, *** denotes p≤0.001; * denotes p value for difference in longitudinal changes.

### METHODS

Participants: Eligibility criteria included age between 10 and 30 years, diagnosis of type 1 diabetes according to ADA criteria within 100 days of starting study drug, presence of at least one islet autoantibody and either random C-peptide>0.3 pmol/mL or C-peptide>0.2 pmol/mL per during a 2 h MMTT. Exclusion criteria included treatment with other immunomodulatory agents, and clinically significant comorbidities including cytopenias, known malignancy, history of thrombosis or LDL cholesterol>155 mg/dL. All participants received standard diabetes management with a target HbA1c of less than 7% in adults and less than 7.5% in participants less than 18 years of age, in accordance with current ADA recommendations.

Randomization and study drug treatment: Participants were randomized in a 2:1 ratio to receive baricitinib 4 mg per day or placebo orally for 48 weeks in blocks of 3, 6 or 9 stratified for age above or below 21 years.

Primary, secondary, and exploratory outcomes: The primary outcome was the average C-peptide during a 2 h MMTT at 48 weeks, calculated as the area under the trapezoidal curve divided by 120 minutes. Secondary outcomes included HbA1c, total daily insulin use, CGM measures from the ambulatory glucose profile. Participants were requested to wear either a Freestyle Libre2 or Dexcom G6 CGM for 14 days at baseline, 12, 24, 48, 72 and 96 weeks. Exploratory mechanistic outcomes included the assessment of basal and cytokine-stimulated phosphorylation of STAT proteins in fresh whole blood, and the frequency of T-cell subsets in PBMC. Samples for these were collected at baseline and 24 weeks.

### Results

Of the 106 children and adults screened, 60 were randomized to receive baricitinib and 31 to receive placebo. The baseline characteristics of the two groups were similar. One participant from each group withdrew before the week 48 assessment due to inability to meet the study requirements.

Primary outcome: After 48 weeks of study drug treatment, median C-peptide was 0.65 (0.31-0.82) pmol/mL/min in the baricitinib group and 0.45 (0.13-0.63) pmol/mL/min in the placebo group (p=0.001). When compared to the baseline measure, C-peptide declined significantly less in the baricitinib treated participants (5.5%) than in placebo treated participants (29.5%, p=0.008). The median C-peptide was also significantly higher in baricitinib compared to placebo treated participants at weeks 12 and 24.

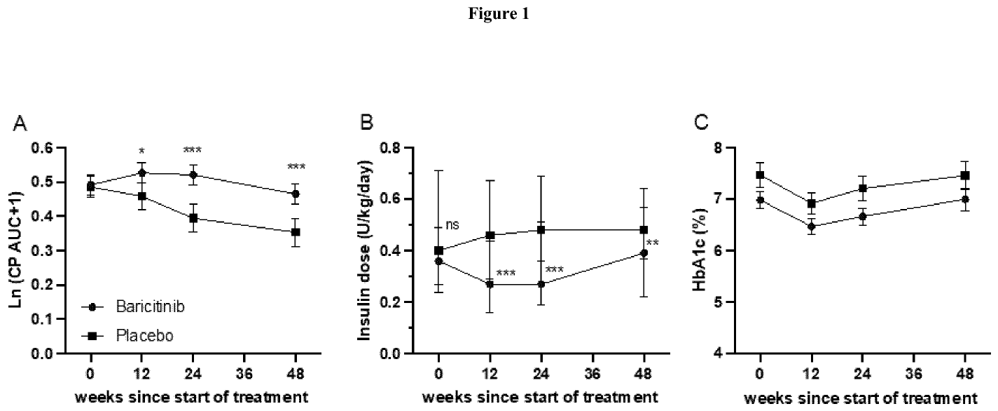

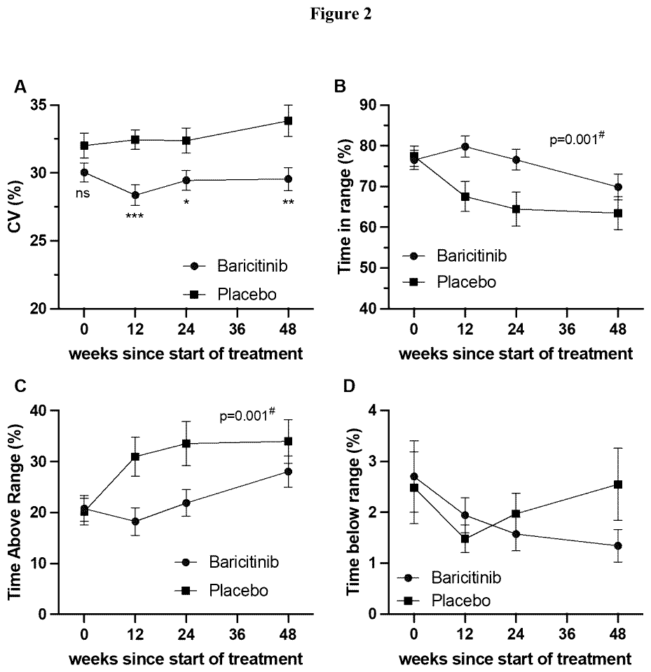

Secondary outcomes: Baricitinib treatment was associated with significantly lower insulin use (FIG. 1B). The daily requirement for the baricitinib participants decreased below baseline at weeks 12 and 24 whereas insulin use for placebo participants increased progressively at each assessment. At week 48, the daily insulin dose was 0.39 (0.22-0.57) U/kg/day and 0.48 (0.37-0.64) U/kg/day in baricitinib and placebo participants respectively (p=0.004). When insulin use was analyzed according to its basal and mealtime components, similar differences to the total were observed between the groups. HbA1c did not differ significantly between groups at weeks 12, 24 or 48 (FIG. 1C). At 48 weeks, HbA1c was 7.0%±0.22 in the baricitinib group and 7.4±0.27 in the placebo group (p=0.216). In contrast, significant improvement in CGM measures were observed in baricitinib participants. Glucose coefficient of variation improved significantly at 12, 24 and 48 weeks (FIG. 2A), whereas percent time above 180 mg/dl (10 mmol/l) (FIG. 2A, 2B) and between 70 and 180 mg/dL (3.9 and 10 mmol/L) was significantly lower and higher respectively in baricitinib treated participants at weeks 12 and 24, but not week 48 (FIG. 2B, 2C). The longitudinal trend across all time points was significant for insulin dose being lower in baricitinib treated participants. Across both groups, participants had very little time below range<70 mg/dl (<3.9 mmol/L) with no significant differences observed between the groups (FIG. 2D).

Safety outcomes: Baricitinib was well tolerated. The frequency and severity of AEs were similar between treatment groups. The median number of AEs was 2 in the baricitinib group and 3 in the placebo group. A total of seven SAEs were recorded, none of which were attributed to study medication. There were no significant differences in measures of lipid profile, liver function or renal function between treatment groups at baseline, 12, 24, or 48 weeks.

The study demonstrates that baricitinib has a positive effect on the preservation of insulin secretion capacity in new-onset type-1 diabetes patients over the course of 48 weeks. This effect was manifested by higher levels of measured C-peptide and lower requirement for insulin administration in the patients treated with baricitinib versus placebo. In addition, baricitinib was well tolerated by the study participants. These results show that oral administration of baricitinib may be employed as a safe, efficacious, and easy to administer disease-modifying agent for the treatment and prevention of new-onset type 1 diabetes.

We claim:

1. A method of treating or preventing new-onset type 1 diabetes in a subject, comprising administering an effective amount of baricitinib, or a pharmaceutically acceptable salt thereof.

2. The method according to claim 1 wherein the new-onset type 1 diabetes is type 1 diabetes clinically diagnosed according to American Diabetes Association guidelines (ADA) within the 100 days prior to treatment commencement.

3. The method according to claim 1 further comprising the administration of an effective amount of insulin, an insulin analogue, and/or other immunomodulatory agents.

4. The method according to claim 1, wherein the amount of baricitinib or a pharmaceutically acceptable salt thereof is 0.5 to 10 mg, 0.5 to 8 mg, 0.5 to 6 mg, 0.5 to 4 mg, 1 to 4 mg, and 2 to 4 mg.

5. The method according to claim 1, wherein the amount of baricitinib or a pharmaceutically acceptable salt thereof is 2 to 4 mg.

6. The method according to claim 1, wherein the amount of baricitinib or a pharmaceutically acceptable salt thereof is selected from 0.5 mg, 1 mg, 1.5 mg, 2 mg, 2.5 mg, 3 mg, 3.5 mg, 4 mg, and 4.5 mg, 5 mg, 5.5 mg, 6 mg, 6.5 mg, 7 mg, 7.5 mg, 8 mg, 8.5 mg, 9 mg, 9.5 mg, and 10 mg.

7. The method according to claim 1, wherein the amount of baricitinib or a pharmaceutically acceptable salt thereof is 1 mg.

8. The method according to claim 1, wherein the amount of baricitinib or a pharmaceutically acceptable salt thereof is 2 mg.

9. The method according to claim 1, wherein the amount of baricitinib or a pharmaceutically acceptable salt thereof is 4 mg.

10. The method according to claim 1, wherein baricitinib or a pharmaceutically acceptable salt thereof is administered orally.

11. The method according to claim 1, wherein baricitinib or a pharmaceutically acceptable salt thereof is administered once daily.

12. The method according to claim 1, wherein baricitinib is administered as the free base.

13. The method according to claim 1, wherein baricitinib or a pharmaceutically acceptable salt thereof is administered as a pharmaceutical composition comprising baricitinib or a pharmaceutical salt thereof with one or more pharmaceutically acceptable carriers, diluents, or excipients.

14. The method according to claim 13, wherein the pharmaceutical composition comprises baricitinib free base.

15. The method according to claim 13, wherein the pharmaceutical composition is in a form selected from a pill, a tablet, a solution, and a suspension.
